(19) United States
(12) Patent Application Publication
(10) Pub. No.: US 2022/0088356 A1
(43) Pub. Date: Mar. 24, 2022

(54) Surgical Instruments Formed from Articulating or Dilating Tubes

(71) Applicant: TransEnterix Surgical, Inc., Morrisville, NC (US)

(72) Inventor: Matthew R Penny, Holly Springs, NC (US)

(21) Appl. No.: 16/513,713

(22) Filed: Jul. 17, 2019

(51) Int. Cl. A61M 29/00; A61B 17/00

(52) U.S. Cl. CPC A61M 29/00; A61B 2017/00327; A61B 2017/00526; A61B 17/00234

(57) ABSTRACT

A shaft for a surgical instrument includes a tubular shaft and an end effector comprising a pair of jaw members. The end effector is formed by laser cutting a distal end of the tubular shaft to form the jaws and to form a pivot. The pivot enables movement of the a jaw about the pivot relative to a more proximal portion of the shaft.

### BACKGROUND

In laparoscopic and robotic surgery, wristed and articulating instruments provide additional dexterity for the surgeon—enabling access to tissue in small, constrained spaces.

Commercially available articulating surgical instruments have overall diameters in the range of 5 to 10 mm and have a variety of mechanical features to enable articulation away from the primary instrument shaft. Some of these instruments include one discreet joint for each articulation direction, while others use multiple joints for a given direction. Articulation is controlled through tendons or rods extending axially through the primary instrument shaft such that a combination of pulling, pushing or rotating the rods or tendons results in movement at the instrument end effector.

There are very some small diameter instruments, also known as “mini-lap” instruments, used in surgery. Mini-lap instruments in use today are fabricated in a 3 mm (or less) diameter that is less invasive to the patient that larger diameter instruments. In fact, some mini-lap instruments do not require surgical incision closure and can be used with percutaneous entry into the abdominal cavity. These instruments are typically “straight-stick” instruments that replicate the functions of their larger, 5 mm counterparts.

There is a strong desire to miniaturize surgical instrumentation as a means to reduce patient tissue trauma. However, size reductions typically result in a reduction in capability and/or an increase in the cost of fabrication. This application describes instrument designs that allow for manufacture of wristed or articulating instruments in a 3 mm outer diameter profile while minimizing the overall impact to cost and capability. This provides surgeons with instruments having excellent dexterity in combination with a diameter that can minimize tissue trauma relative to more conventionally-sized instruments.

### BRIEF DESCRIPTION OF THE DRAWINGS

FIG. 1 is a model of outer tube showing pivot cut for the dilation component of the Articulating and Dilating Tube.

FIGS. 2A and 2B show an articulating and dilating tube forming a surgical instrument, with the jaw elements in the opened and closed positions, respectively.

FIG. 3A shows the tube assembled with an inner core and tendons for articulation and/or jaw actuation. FIG. 3B is similar but shows only the inner core, tendons, and the tendon fixation point.

FIGS. 4A-C show fenestrated, rat-tooth, and wavy jaw profiles, respectively, for the outer tube of the instrument.

### DETAILED DESCRIPTION

This application shows and describes surgical instruments having instrument shafts shafts and end effectors positioned at the distal end of an instrument shaft. The surgical instruments may be ones configured for manual operation using a proximal drive mechanism in the form of a manually actuated instrument handle at the proximal end of the instrument shaft. Alternatively, for robotically assisted operation, the instrument's proximal drive mechanism receives motion from robotically controlled actuators operating in accordance with surgeon input to a surgical robotic system.

The described embodiments make use of laser cut tubing technology to create the pivots of the shaft in-line with the outer diameter. This will maximize the internal diameters of the instruments, which will be occupied with tendons to create the articulating motion.

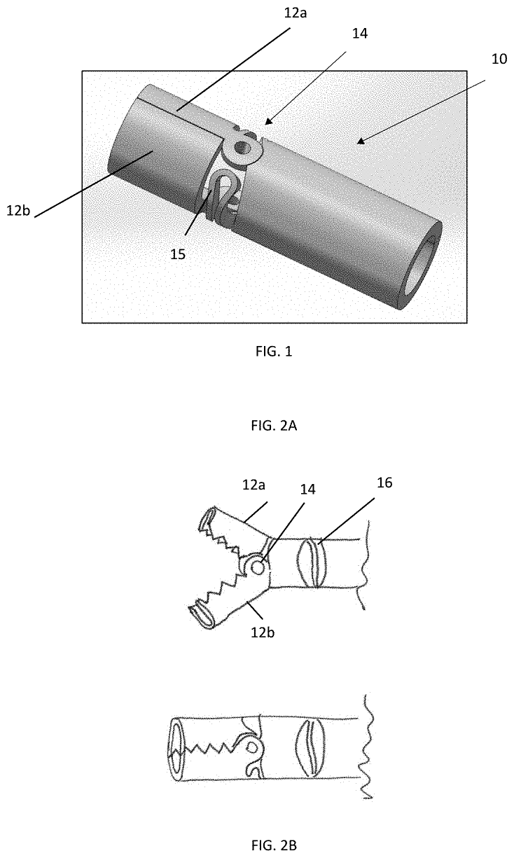

FIG. 1 shows an articulating and dilating tube 10. The tube 10 is cut to form a pair of distal jaw members 12a, 12b, each having a distal joint or pivot 14 that is cut for the dilation component of the articulating and dilating tube. The specific design of the pivots can vary. In this embodiment, for a given jaw member 12a, there is a first pivot contact on the jaw member 12a that is in the form of a convex surface or male part and a second pivot contact on the more proximal part of the shaft, that is in the form of a concave surface or female part. The jaw member 12a and the part of the tube 10 proximal to the pivot 14 remain integrally connected by means of at least one connecting member 15 laser cut into the tube. In preferred embodiments, the connecting member 15 is a spring member, such as a leaf spring. Each connecting member extends between a jaw member 12a and the more proximal part of the shaft as shown.

The distal joint is designed to allow the distal-most segment of the tube (comprising the jaw members) to not only angle about the pivot point at pivot 14, but also to dilate about the same pivot point. This is because the distal-most segment is split along its axis—enabling one section to bend in different directions and at different amounts than the opposite section. The dilation is a flowering type of dilation as opposed to a more symmetrical type of dilation that occurs when a stent expands Referring to FIGS. 4A-4C, the distal end of the tube 10 may be cut with a saw-tooth shape, or other desired end effector tooth profiles, up to the jaw pivot. Each side of the end effector tooth profile cut is combined with one surface of the remaining pivot. As discussed above, a spring cut behind each jaw and between the two pivots retains the jaw component onto the more proximal part of the tube 10. When assembled with the tendons, the jaw segments can move together in the same direction about the pivots, or away from one another, depending on the combination of tendons pulled.

Co-pending application Ser. No. 15/003,656, incorporated herein by reference, describes articulating tubes fabricated with laser cutting methods. In those embodiments, multiple joints are laser cut into to the tube. These joints allow the tube to bend about an axis where the distal portion of the bend and the proximal portion of the bend are still tubular in nature. FIGS. 2A and 2B show one such joint 16 that works on this principle laser cut into the tube 10 which, as shown, may also have the dilating joint 14 described above.

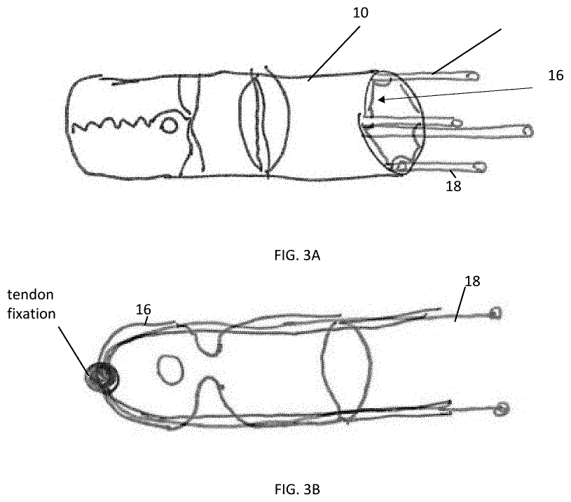

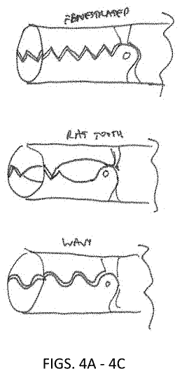

Note that as described in Co-pending application Ser. No. 15/003,656, the instrument shaft may include an inner core 16 (FIGS. 3A and 3B) or tube within the lumen of the tube 10. Configurations for the inner core or tube member that may be adapted for the embodiments described here are described in the co-pending application. The internal core can be made of plastic or metal and may be fixed at specific locations to the outer tube 10. The tendon paths may be fully contained within the inner core, or they may be defined by the outer profile of the inner core and the inner diameter of the articulating and dilating tube.

As shown in FIGS. 3A and 3B, the tendons 18 for articulation and jaw actuation will mate to the inner core 16 in such a way as to transmit forces exerted on the proximal end of the tendon to the articulating and dilating tube structure by way of the fixations between the inner core and the outer tube structure.

The ability to cut different jaw shapes into the outer tube with a laser could reduce the overall cost of the instrument and enable the inner core to be a common part amongst all instruments. Alternatively, components of the jaw profiles could also exist on the inner core if desired. This may open up opportunities to create multi-function instruments where the external tube structure can have one purpose and the inner core can serve a second purpose.

The described instrument shaft design is beneficial in that it enables a very small diameter instrument (e.g. one with a 3 mm outer diameter) to be configured to articulate by maximizing the space within the outer tube lumen so that tendons may pass through the lumen. It may also decrease the costs for machining surgical end effectors by using laser cutting technology, and allow for a reduction in the distance from the articulation joint to the end effector tip compared with other instrument designs.

What is claimed is:

1. A shaft for a surgical instrument comprising:
a tubular shaft and an end effector comprising a pair of jaw members, the end effector formed by laser cutting a distal end of the tubular shaft to form the jaws and to form a pivot, the pivot enabling movement of the a jaw about the pivot relative to a more proximal portion of the shaft.

2. The shaft of claim 1, further including a joint laser cut into the shaft, proximal to the pivot, the joint enabling articulation of the end effector relative to the shaft.

3. The shaft of claim 1, wherein the shaft further includes an inner tubular member having an outer surface, wherein the tubular member is an outer tubular member coaxially disposed over the inner tubular member, defining tendon pathways between the outer surface of the inner tubular member and the inner surface of the outer tubular member, and wherein the shaft further includes actuation elements disposed within the channels, each actuation element having a distal end anchored to the shaft and a proximal end.

4. A method of making a surgical instrument shaft, comprising the steps of:
providing an elongate tube;
laser cutting a pair of jaws into the elongate tube to create a distal end effector on a proximal portion of the tube;
laser cutting a pivot between each jaw member and the proximal portion of the tube.

5. The method of claim 5, further comprising the step of laser cutting an articulation joint in the proximal portion of the tube.
